(19) United States
(12) Patent Application Publication
(10) Pub. No.: US 2019/0365453 A1
(43) Pub. Date: Dec. 5, 2019

(54) SYSTEM AND METHOD FOR LIMITING DIFFERENTIAL PRESSURE ACROSS PROPORTIONAL VALVE DURING CRYOABLATION PROCEDURES

(71) Applicant: Boston Scientific Scimed, Inc., Maple Grove, MN (US)

(72) Inventors: Chadi Harmouche, Saint Laurent (CA); Eric Ryba, San Diego, CA (US)

(21) Appl. No.: 16/540,412

(22) Filed: Aug. 14, 2019

Related U.S. Application Data
(63) Continuation of application No. PCT/US2018/018017, filed on Feb. 13, 2018.
(60) Provisional application No. 62/460,687, filed on Feb. 17, 2017.

(51) Int. Cl. A61B 18/02; A61B 18/14

(52) U.S. Cl. CPC A61B 18/02; A61B 2018/00577; A61B 18/1492

(57) ABSTRACT

A differential pressure limiter (226) for limiting a differential fluid pressure of cryogenic fluid (228) during a cryoablation procedure includes one or more of a fluid source (216), a pressure regulator (230), one or more proportional valves (232) and a backpressure regulator (234). The fluid source (216) selectively retains cryogenic fluid (228). The pressure regulator (230) receives cryogenic fluid (228) from the fluid source (216) and regulates a fluid pressure of the cryogenic fluid (228). The proportional valve(s) (232) receives cryogenic fluid (228) from the pressure regulator (230) and at least partially controls a flow rate of the cryogenic fluid (228). The backpressure regulator (234) receives cryogenic fluid (228) from the proportional valve(s) (232) and manually and/or automatically changes and/or decreases the differential fluid pressure of the cryogenic fluid (228) across the proportional valve(s) (232) and/or limits the differential fluid pressure across the proportional valve(s) (232) within a predetermined range.

### CROSS-REFERENCE TO RELATED APPLICATION

This application is a continuation of International Application No. PCT/US2018/018017, with an international filing date of Feb. 13, 2018, which claims the benefit of U.S. Provisional Application No. 62/460,687, filed on Feb. 17, 2017, and entitled “SYSTEM AND METHOD FOR LIMITING DIFFERENTIAL PRESSURE ACROSS PROPORTIONAL VALVE DURING CRYOABLATION PROCEDURES”. As far as permitted, the contents of International Application No. PCT/US2018/018017 and U.S. Provisional Application Nos. 62/460,687 are incorporated herein by reference for all purposes.

### BACKGROUND

Cardiac arrhythmias involve an abnormality in the electrical conduction of the heart and are a leading cause of stroke, heart disease, and sudden cardiac death. Treatment options for patients with arrhythmias include medications, implantable devices, and catheter ablation of cardiac tissue.

Catheter ablation involves delivering ablative energy to tissue inside the heart to block aberrant electrical activity from depolarizing heart muscle cells out of synchrony with the heart's normal conduction pattern. The procedure is performed by positioning a portion, such as a tip, of an energy delivery catheter adjacent to diseased or targeted tissue in the heart. One form of energy that is used to ablate diseased heart tissue includes cryogenics (also referred to herein as “cryoablation”). During this procedure, the tip of the catheter is positioned adjacent to target cardiac tissue, at which time energy is delivered to create tissue necrosis, rendering the ablated tissue incapable of conducting electrical signals.

The dose of the energy delivered is an important factor in increasing the likelihood that the treated tissue is permanently incapable of conduction. At the same time, delicate collateral tissue, such as the esophagus, the bronchus, and the phrenic nerve surrounding the ablation zone can be damaged and can lead to undesired complications. Thus, the operator must finely balance delivering therapeutic levels of energy to achieve intended tissue necrosis while avoiding excessive energy leading to collateral tissue injury.

Atrial fibrillation is one of the most common arrhythmias treated using cryoablation. In the earliest stages of the disease, paroxysmal atrial fibrillation, the treatment strategy involves isolating the pulmonary veins from the left atrial chamber. Recently, the use of techniques known as “balloon cryotherapy” catheter procedures to treat atrial fibrillation have increased. During the balloon cryotherapy procedure, a cryogenic fluid (such as nitrous oxide, or any other suitable fluid) is delivered under pressure to an interior of one or more cryogenic balloons which are positioned against the target tissue. Using this method, the extremely frigid fluid causes necrosis of the target tissue, thereby rendering the ablated tissue incapable of conducting unwanted electrical signals.

One or more proportional valves are used to control the pressure and/or flow of the cryogenic fluid that flows to the cryogenic balloon. During cryoablation procedures, relatively high injection pressures are required, leading to potentially broad ranges of differential fluid pressures across the proportional valves. These wide ranging fluid pressures can cause erratic and/or inconsistent temperatures of the cryogenic balloon, and can result in undesirable outcomes for the cryoablation procedure. Currently there is a significant challenge to manufacture cost-effective proportional valves capable of handling a wide range of differential fluid pressures across the input and output ports of the proportional valves. Although customized proportional valves which can handle these wide-ranging differential fluid pressures are technically available, these types of custom proportional valves can be relatively complex, resulting in possible reliability issues. Further, these custom proportional valves can require long development lead times, and can be very costly to develop and manufacture.

### SUMMARY

The present invention is directed toward a differential pressure limiter for limiting a differential fluid pressure of a cryogenic fluid that is delivered to a catheter system during a cryoablation procedure. In certain embodiments, the differential pressure limiter can include one or more of a fluid source, a pressure regulator, a first proportional valve and a backpressure regulator. The fluid source selectively retains the cryogenic fluid. The pressure regulator receives the cryogenic fluid from the fluid source. The pressure regulator can also regulate a fluid pressure of the cryogenic fluid. In some embodiments, the pressure regulator can include a regulator input, a regulator output and/or a control valve. In such embodiments, the control valve can reduce an input pressure of the cryogenic fluid at the regulator input to a desired output pressure at the regulator output.

In certain embodiments, the first proportional valve receives the cryogenic fluid from the pressure regulator. Further, the first proportional valve at least partially controls a flow rate of the cryogenic fluid to the catheter system during the cryoablation procedure. In some embodiments, the fluid pressure of the cryogenic fluid delivered to the first proportional valve from the pressure regulator is less than the fluid pressure of the cryogenic fluid within the fluid source. In other embodiments, the first proportional valve can include a first proportional valve input and/or a first proportional valve output. In such other embodiments, the first proportional valve can change the flow rate and/or fluid pressure of the cryogenic fluid at the first proportional valve output in a proportional manner to the flow rate and/or fluid pressure of the cryogenic fluid at the first proportional valve input. In other embodiments, the cryogenic fluid can have a first differential fluid pressure across the first proportional valve.

In various embodiments, the backpressure regulator is in fluid communication with the first proportional valve so that the first proportional valve is positioned between the pressure regulator and the backpressure regulator. The backpressure regulator can decrease the first differential fluid pressure of the cryogenic fluid across the first proportional valve.

In some embodiments, the backpressure regulator decreases the first differential fluid pressure of the cryogenic fluid across the first proportional valve to less than approximately 100 psi, 50 psi, 30 psi, or 10 psi.

In various embodiments, the backpressure regulator maintains the first differential fluid pressure across the first proportional valve within a predetermined range.

In one embodiment, the differential pressure limiter also includes a second proportional valve that is positioned between the pressure regulator and the backpressure regulator.

In certain embodiments, the backpressure regulator can be manually adjustable to change the first differential fluid pressure of the cryogenic fluid across the first proportional valve. Alternatively, the backpressure regulator can be automatically adjustable to change the first differential fluid pressure of the cryogenic fluid across the first proportional valve.

In certain other embodiments, the differential pressure limiter includes a fluid source, a pressure regulator, a first proportional valve and a second proportional valve. The fluid source selectively retains the cryogenic fluid. The pressure regulator receives the cryogenic fluid from the fluid source. Further, in some embodiments, the pressure regulator can have a regulator output. The pressure regulator can regulate fluid pressure of the cryogenic fluid at the regulator output. In various embodiment, the first proportional valve receives the cryogenic fluid from the regulator output of the pressure regulator. The cryogenic fluid can have a first differential fluid pressure across the first proportional valve. The second proportional valve at least partially controls a flow rate of the cryogenic fluid to the catheter system during the cryoablation procedure. Further, in certain embodiments, the second proportional valve receives the cryogenic fluid from the first proportional valve and can change the differential fluid pressure of the cryogenic fluid from the first differential fluid pressure to a second differential fluid pressure that is less than the first differential fluid pressure.

In various embodiments, the second proportional valve decreases the second differential fluid pressure of the cryogenic fluid across the second proportional valve based on the first differential fluid pressure across the first proportional valve.

In some embodiments, the differential pressure limiter further includes a third proportional valve that at least partially controls the flow rate of the cryogenic fluid to the catheter system during the cryoablation procedure. The third proportional valve receives the cryogenic fluid from the second proportional valve and can change the differential fluid pressure of the cryogenic fluid from the second differential fluid pressure to a third differential fluid pressure that is less than the second differential fluid pressure.

In certain embodiments, the second proportional valve is positioned between the first proportional valve and the third proportional valve.

In some embodiments, the third proportional valve decreases the third differential fluid pressure of the cryogenic fluid across the third proportional valve based on the second differential fluid pressure across the second proportional valve.

The present invention is also directed toward a method that includes the step of delivering a cryogenic fluid to a catheter system via a differential pressure limiter that is configured so that a proportional valve is positioned between a pressure regulator and a backpressure regulator to limit a differential fluid pressure of the cryogenic fluid across the proportional valve during a cryoablation procedure.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic side view of a patient and one embodiment of a cryogenic balloon catheter assembly including a differential pressure limiter having features of the present invention;

FIG. 2 is a schematic side view of one embodiment of the differential pressure limiter; and

FIG. 3 is a schematic side view of another embodiment of the differential pressure limiter.

### DESCRIPTION

Embodiments of the present invention are described herein in the context of a differential pressure limiter. Those of ordinary skill in the art will realize that the following detailed description of the present invention is illustrative only and is not intended to be in any way limiting. Other embodiments of the differential pressure limiter will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

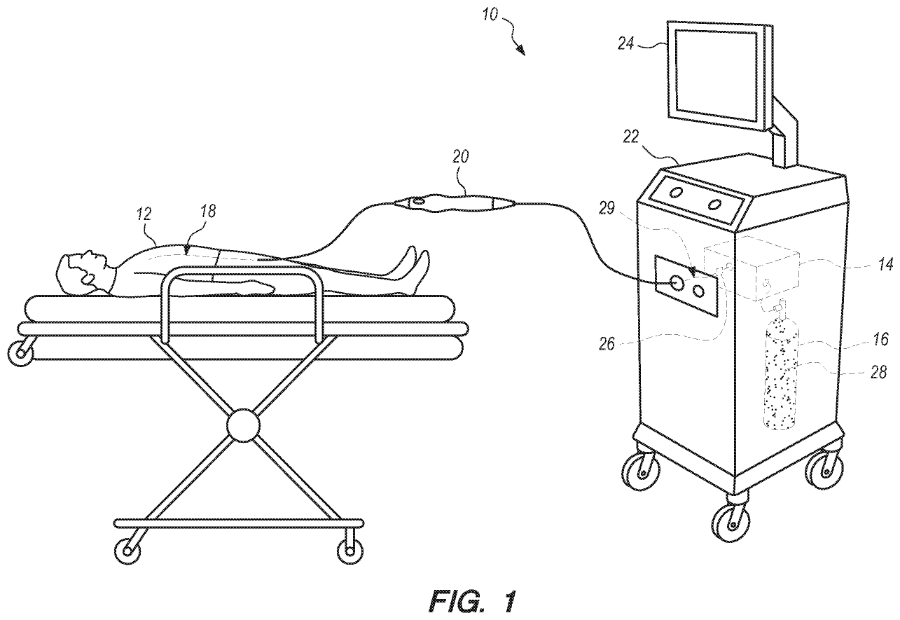

FIG. 1 is a schematic side view of one embodiment of a cryogenic balloon catheter system 10 (also sometimes referred to herein as a “catheter system”) for use with a patient 12, which can be a human being or an animal. Although the catheter system 10 is specifically described herein with respect to a cryogenic balloon catheter system, it is understood and appreciated that other types of catheter systems and/or ablation systems can equally benefit by the teachings provided herein. For example, in certain non-exclusive alternative embodiments, the present invention can be equally applicable for use with any suitable types of ablation systems and/or any suitable types of catheter systems. Thus, the specific reference herein to use as part of a cryogenic balloon catheter system is not intended to be limiting in any manner.

The design of the catheter system 10 can be varied. In certain embodiments, such as the embodiment illustrated in FIG. 1, the catheter system 10 can include one or more of a control system 14, a fluid source 16, a balloon catheter 18, a handle assembly 20, a control console 22, a graphical display 24 and a differential pressure limiter 26. It is understood that although FIG. 1 illustrates the structures of the catheter system 10 in a particular position, sequence and/or order, these structures can be located in any suitably different position, sequence and/or order than that illustrated in FIG. 1. It is also understood that the catheter system 10 can include fewer or additional components than those specifically illustrated and described herein.

In various embodiments, the control system 14 is configured to monitor and control the various processes of the ablation procedure. More specifically, the control system 14 can control release and/or retrieval of a cryogenic fluid 28 to and/or from the balloon catheter 18. In certain embodiments, the control system 14 can control various structures described herein that are responsible for maintaining and/or adjusting a flow rate and/or fluid pressure of the cryogenic fluid 28 that is released to the balloon catheter 18 during a cryoablation procedure. In such embodiments, the catheter system 10 delivers ablative energy in the form of cryogenic fluid 28 to cardiac tissue of the patient 12 to create tissue necrosis, rendering the ablated tissue incapable of conducting electrical signals. Additionally, in various embodiments, the control system 14 can control activation and/or deactivation of one or more other processes of the balloon catheter 18 described herein. Further, or in the alternative, the control system 14 can receive data and/or other information (hereinafter sometimes referred to as “sensor output”) from various structures within the catheter system 10. In some embodiments, the control system 14 can assimilate and/or integrate the sensor output, and/or any other data or information received from any structure within the catheter system 10. Additionally, or in the alternative, the control system 14 can control positioning of portions of the balloon catheter 18 within the body of the patient 12, and/or can control any other suitable functions of the balloon catheter 18.

The fluid source 16 contains the cryogenic fluid 28, which is delivered to the balloon catheter 18 with or without input from the control system 14 during the cryoablation procedure. The type of cryogenic fluid 28 that is used during the cryoablation procedure can vary. In one non-exclusive embodiment, the cryogenic fluid 28 can include liquid nitrous oxide. In another non-exclusive embodiment, the cryogenic fluid 28 can include liquid nitrogen. However, any other suitable cryogenic fluid 28 can be used.

The design of the balloon catheter 18 can be varied to suit the specific design requirements of the catheter system 10. As shown, the balloon catheter 18 is inserted into the body of the patient 12 during the cryoablation procedure. In one embodiment, the balloon catheter 18 can be positioned within the body of the patient 12 using the control system 14. Stated in another manner, the control system 14 can control positioning of the balloon catheter 18 within the body of the patient 12. Alternatively, the balloon catheter 18 can be manually positioned within the body of the patient 12 by a health care professional (also sometimes referred to herein as an “operator”). As used herein, health care professional and/or operator can include a physician, a physician's assistant, a nurse and/or any other suitable person and/or individual. In certain embodiments, the balloon catheter 18 is positioned within the body of the patient 12 utilizing at least a portion of the sensor output received from the balloon catheter 18. For example, in various embodiments, the sensor output is received by the control system 14, which can then provide the operator with information regarding the positioning of the balloon catheter 18. Based at least partially on the sensor output feedback received by the control system 14, the operator can adjust the positioning of the balloon catheter 18 within the body of the patient 12 to ensure that the balloon catheter 18 is properly positioned relative to targeted cardiac tissue. While specific reference is made herein to the balloon catheter 18, as noted above, it is understood that any suitable type of medical device and/or catheter may be used.

The handle assembly 20 is handled and used by the operator to operate, position and/or control the balloon catheter 18. The design and specific features of the handle assembly 20 can vary to suit the specific design requirements of the catheter system 10. In the embodiment illustrated in FIG. 1, the handle assembly 20 is separate from, but in electrical and/or fluid communication with the control system 14, the fluid source 16 and/or the graphical display 24. In some embodiments, the handle assembly 20 can integrate and/or include at least a portion of the control system 14 within an interior of the handle assembly 20. It is understood that the handle assembly 20 can include additional components than those specifically illustrated and described herein.

In the embodiment illustrated in FIG. 1, the control console 22 includes at least a portion of the control system 14, the fluid source 16, the graphical display 24 and the differential pressure limiter 26. However, in alternative embodiments, the control console 22 can contain additional structures not shown or described herein. Still alternatively, the control console 22 may not include various structures that are illustrated within the control console 22 in FIG. 1. For example, in one embodiment, the control console 22 does not include the graphical display 24.

In various embodiments, the graphical display 24 is electrically connected to the control system 14. Additionally, the graphical display 24 provides the operator of the catheter system 10 with information that can be used before, during and after the cryoablation procedure. For example, the graphical display 24 can provide the operator with information based on the sensor output, and any other relevant information that can be used before, during and after the cryoablation procedure. The specifics of the graphical display 24 can vary depending upon the design requirements of the catheter system 10, or the specific needs, specifications and/or desires of the operator.

In one embodiment, the graphical display 24 can provide static visual data and/or information to the operator. In addition, or in the alternative, the graphical display 24 can provide dynamic visual data and/or information to the operator, such as video data or any other data that changes over time. Further, in various embodiments, the graphical display 24 can include one or more colors, different sizes, varying brightness, etc., that may act as alerts to the operator. Additionally, or in the alternative, the graphical display 24 can provide audio data or information to the operator.

The differential pressure limiter 26 maintains, controls and/or limits a differential fluid pressure of the cryogenic fluid 28 delivered to the catheter system 10. The design of the differential pressure limiter 26 can be varied depending upon the specific design requirements of the catheter assembly 10. In various embodiments, the control system 14 can control activation and/or deactivation of one or more processes of the differential pressure limiter 26 described herein. In the embodiment illustrated in FIG. 1, the differential pressure limiter 26 can be integrated, included and/or positioned within the control console 22. The differential pressure limiter 26 can be positioned at any location within the control console 22. In other embodiments, the differential pressure limiter 26 may not be integrated, included and/or positioned within the control console 22. The differential pressure limiter 26 can be positioned at any location outside the control console 22. Additionally, and/or alternatively, the differential pressure limiter 26 can be integrated, included and/or positioned within any other suitable structure of the catheter system 10.

In certain embodiments, the catheter system 10 and/or the differential pressure limiter 26 may include one or more conduits 29, cables or other means of transferring fluids or electrical signals. In the embodiment illustrated in FIG. 1, the conduits 29 connect the differential pressure limiter 26 with the fluid source 16 and/or the handle assembly 20. In this embodiment, the conduits 29 can allow the flow of cryogenic fluid 28 from the fluid source 216 to the handle assembly 20 and ultimately to the balloon catheter 18 that is positioned within the patient 12. In certain embodiments, the conduits 29 can include relatively small diameter tubes through which the cryogenic fluid 28 flows and/or moves. Alternatively, the conduits 29 may include any other suitable design.

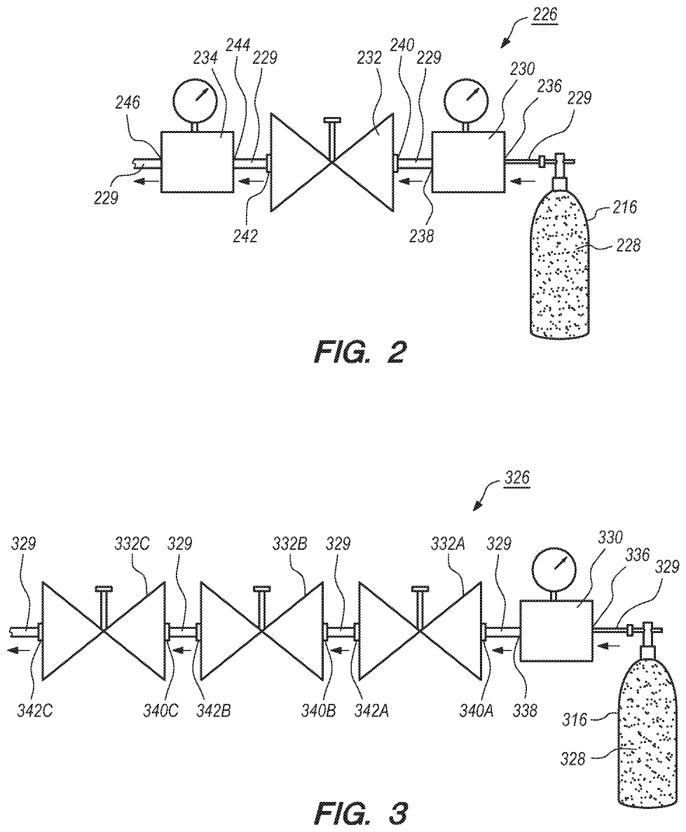

FIG. 2 is a schematic side view of one embodiment of the differential pressure limiter 226. In the embodiment illustrated in FIG. 2, the differential pressure limiter 226 includes the fluid source 216 that selectively contains the cryogenic fluid 228, one or more conduits 229, a pressure regulator 230, one or more proportional valves 232 (only one, a first proportional valve 232, is illustrated in FIG. 2) and a backpressure regulator 234. It is understood that the differential pressure limiter 226 can include fewer or additional components than those specifically illustrated and described herein. In certain embodiments, the differential pressure limiter 226 limits the differential fluid pressure across the first proportional valve 232 to be maintained within a predetermined range. In other words, with this design, relatively high injection pressures, which normally may have a relatively wide differential fluid pressure range, can be better controlled and kept within a narrower operating range.

The fluid source 216 contains the cryogenic fluid 228 that is sent to the balloon catheter 18 (illustrated in FIG. 1) by the control system 14 (illustrated in FIG. 1) during the cryoablation procedure. Once the cryoablation procedure has initiated, the cryogenic fluid 228 can be delivered and, the resulting gas after a phase change, can be retrieved from the balloon catheter 18, and can either be vented, discarded or re-retained by the fluid source 216.

The pressure regulator 230 receives the cryogenic fluid 228 from the fluid source 216. The specific type of pressure regulator 230 can be varied; however, any suitable pressure regulator 230 could be utilized in the differential pressure limiter 226. In various embodiments, the pressure regulator 230 can include a control valve that reduces an input pressure of the cryogenic fluid 228 at a regulator input 236 to a desired output pressure at a regulator output 238. The pressure regulator 230 can have an output pressure setting, a restrictor and/or a sensor all in the one body, and/or it can include a separate pressure sensor, controller and flow valve. Further, the pressure regulator 230 provides an avenue to deliver the cryogenic fluid 228 to the proportional valve 232 at a fluid pressure that is less than the fluid pressure within the fluid source 216.

The first proportional valve 232 includes a valve that changes the fluid pressure and/or flow rate of the cryogenic fluid 228 at a first proportional valve output 242 in a proportional manner to the fluid pressure and/or flow rate of the cryogenic fluid 228 at a first proportional valve input 240. Further, the cryogenic fluid 228 may have a first differential fluid pressure across the first proportional valve. The specific type of first proportional valve 232 can be varied; however, any suitable proportional valve 232 could be utilized in the differential pressure limiter 226. In the embodiment illustrated in FIG. 2, the first proportional valve is positioned between the pressure regulator 230 and the backpressure regulator 234 so that the cryogenic fluid 228 flows through the pressure regulator 230 to the first proportional valve 232, then to the backpressure regulator 234, and eventually to the handle assembly 20 and/or balloon catheter 18, all via one or more conduits 229. Additionally, and/or in the alternative, it is understood that although one proportional valve 232 is illustrated in FIG. 2, any suitable number of proportional valves may be used, i.e., a second proportional valve, a third proportional valve, etc.

The backpressure regulator 234 receives the cryogenic fluid 228 from the first proportional valve 232. In various embodiments, the backpressure regulator 234 can include a control valve that regulates the upstream fluid pressure, thereby controlling the first differential fluid pressure across the first proportional valve 232 by opening up only as much as necessary to hold back the desired fluid pressure at the backpressure valve inlet 244 (upstream) to the backpressure regulator 234. The backpressure regulator 234 can also include a backpressure valve outlet 246. The specific type of backpressure regulator 234 can be varied; however, any suitable backpressure regulator 234 could be utilized in the differential pressure limiter 226.

In one embodiment, the backpressure regulator 234 can decrease the first differential fluid pressure of the cryogenic fluid 228 across the first proportional valve 232 to the predetermined range that can be set by the operator of the differential pressure limiter 226. In various embodiments, the predetermined range can vary such that the first differential fluid pressure is kept within a more narrow operating range. For example, the predetermined range can include a first differential fluid pressure of greater than approximately 10 psi and less than approximately 100 psi. In another embodiment, the backpressure regulator 234 can decrease the first differential fluid pressure of the cryogenic fluid 228 across the first proportional valve 232 to less than approximately 100 psi. In non-exclusive alternative embodiments, the backpressure regulator 234 can decrease the first differential fluid pressure of the cryogenic fluid 228 across the first proportional valve 232 to less than approximately 50 psi, 30 psi or 10 psi. Still alternatively, the backpressure regulator 234 can decrease the first differential fluid pressure of the cryogenic fluid 228 across the first proportional valve 232 to various values outside of the foregoing ranges.

In certain embodiments, the backpressure regulator 234 or any other components of the differential pressure limiter 226 can be manually adjustable by the operator of the differential pressure limiter 226 to change the first differential fluid pressure of the cryogenic fluid 228 across the first proportional valve 232. Alternatively, the backpressure regulator 234 or any other components of the differential pressure limiter 226 can be automatically adjustable to change the first differential fluid pressure of the cryogenic fluid 228 across the first proportional valve 232.

With this design, the use of the backpressure regulator 234 can allow for the first differential fluid pressure across the first proportional valve 232 to be set to a relatively low level, which allows for a more reliable and a higher precision flow control.

FIG. 3 is a schematic side view of another embodiment of the differential pressure limiter 326. In the embodiment illustrated in FIG. 3, the differential pressure limiter 326 includes the fluid source 316 that selectively contains the cryogenic fluid 328, one or more conduits 329, the pressure regulator 330, and one or more proportional valves 332 (three proportional valves, including a first proportional valve 332A, a second proportional valve 332B, and a third proportional valve 332C, are illustrated in FIG. 3, in one non-exclusive embodiment). The differential pressure limiter 326 limits the differential fluid pressure across the proportional valves 332A, 332B, 332C, to be maintained within the predetermined range. It is understood that although three proportional valves 332A, 332B, 332C, are illustrated in FIG. 3, any suitable number of proportional valves may be used, which may exceed or be fewer than three. With these designs, relatively high injection fluid pressures, which normally may have a relatively wide differential fluid pressure range, can be better controlled and kept within a narrower, safer operating range.

The fluid source 316 contains the cryogenic fluid 328 that is sent to the balloon catheter 18 (illustrated in FIG. 1) by the control system 14 (illustrated in FIG. 1) during the cryoablation procedure. Once the cryoablation procedure has initiated, the cryogenic fluid 328 can be delivered and, the resulting gas after a phase change, can be retrieved from the balloon catheter 18, and can either be vented, discarded or re-retained by the fluid source 316.

The pressure regulator 330 receives the cryogenic fluid 328 from the fluid source 316. In various embodiments, the pressure regulator 330 can include a control valve that reduces the input pressure of the cryogenic fluid 328 at the regulator input 336 to the desired output pressure of the cryogenic fluid 328 at the regulator output 338. The pressure regulator 330 can have an output pressure setting, a restrictor and/or a sensor all in the one body, and/or it can include a separate pressure sensor, controller and/or flow valve. Further, the pressure regulator 330 provides an avenue to deliver the cryogenic fluid 328 to the one or more proportional valves 332A, 332B, and/or 332C, at the fluid pressure that is less than the fluid pressure within the fluid source 316.

Each proportional valve 332A, 332B, 332C, can include a valve that changes the fluid pressure and/or flow rate of the cryogenic fluid 328 at a proportional valve output 342A, 342B, 342C, respectively, in a proportional manner to the fluid pressure and/or flow rate of the cryogenic fluid 328 at a proportional valve input 340A, 340B, 340C, respectively. Additionally, in certain embodiments, the cryogenic fluid 328 may have a first differential fluid pressure across the first proportional valve 332A, a second differential fluid pressure across the second proportional valve 332B and/or a third differential fluid pressure across the third proportional valve 332C.

In the embodiment illustrated in FIG. 3, the proportional valves 332A, 332B, 332C, are aligned in series so that the cryogenic fluid 328 flows through the pressure regulator 330 to the first proportional valve 332A, then to the second proportional valve 332B, then to the third proportional valve 332C, and eventually to the handle assembly 20 (illustrated in FIG. 1) and/or balloon catheter 18, all via one or more conduits 329. In this embodiment, the second proportional valve 332B is positioned between the first proportional valve 332A and the third proportional valve 332C. However, while FIG. 3 illustrates the proportional valves 332A, 332B, 332C, in a particular position, sequence and/or order, the proportional valves 332A, 332B, 332C can be located in any suitably different position, sequence and/or order than that illustrated in FIG. 3. Further, it is recognized that the “first proportional valve 332A,” the “second proportional valve 332B” and the “third proportional valve 332C” can be used interchangeably.

Additionally, in the embodiment illustrated in FIG. 3, the plurality of proportional valves 332A, 332B, 332C, can stage-wise decrease the differential fluid pressure of the cryogenic fluid 328 while maintaining and/or controlling upstream differential fluid pressure. For example, in various embodiments, the second proportional valve 332B can receive the cryogenic fluid 328 from the first proportional valve 332A and change the differential fluid pressure of the cryogenic fluid 328 from the first differential fluid pressure to the second differential fluid pressure that is less than the first differential fluid pressure. In other embodiments, the second proportional valve 332B can decrease the second differential fluid pressure of the cryogenic fluid 328 across the second proportional valve 332B based on the first differential fluid pressure across the first proportional valve 332A. In various embodiments, the third proportional valve 332C can then receive the cryogenic fluid 328 from the second proportional valve 332B and change the differential fluid pressure of the cryogenic fluid 328 from the second differential fluid pressure to the third differential fluid pressure that is less than the second differential fluid pressure. In other embodiments, the third proportional valve 332C can decrease the third differential fluid pressure of the cryogenic fluid 328 across the third proportional valve 332C based on the second differential fluid pressure across the second proportional valve 332B.

With the designs shown and described herein, the need for costly and complex custom proportional valves is obviated. Further, existing proportional valves can be used well within their defined operating range. It is understood that although a number of different embodiments of the differential pressure limiter have been illustrated and described herein, one or more features of any one embodiment can be combined with one or more features of one or more of the other embodiments, provided that such combination satisfies the intent of the present invention.

While a number of exemplary aspects and embodiments of the differential pressure limiter have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

We claim:

1. A differential pressure limiter for limiting a differential fluid pressure of a cryogenic fluid that is delivered to a catheter assembly from a fluid source during a cryoablation procedure, the differential pressure limiter comprising:
a pressure regulator that receives the cryogenic fluid from the fluid source, the pressure regulator regulating a fluid pressure of the cryogenic fluid;
a first proportional valve that receives the cryogenic fluid from the pressure regulator, the cryogenic fluid having a first differential fluid pressure across the first proportional valve, the first proportional valve at least partially controlling a flow rate of the cryogenic fluid to the catheter assembly during the cryoablation procedure; and
a backpressure regulator that is in fluid communication with the first proportional valve so that the first proportional valve is positioned between the pressure regulator and the backpressure regulator, the backpressure regulator decreasing the first differential fluid pressure of the cryogenic fluid across the first proportional valve.

2. The differential pressure limiter of claim 1 wherein the fluid pressure of the cryogenic fluid delivered to the first proportional valve from the pressure regulator is less than the fluid pressure of the cryogenic fluid within the fluid source.

3. The differential pressure limiter of claim 1 wherein the pressure regulator includes a control valve.

4. The differential pressure limiter of claim 3 wherein the pressure regulator includes a regulator input and a regulator output, and wherein the control valve reduces an input pressure of the cryogenic fluid at the regulator input to a desired output pressure at the regulator output.

5. The differential pressure limiter of claim 1 wherein the first proportional valve includes a first proportional valve input and a first proportional valve output, the first proportional valve changing the flow rate of the cryogenic fluid at the first proportional valve output in a proportional manner to the flow rate of the cryogenic fluid at the first proportional valve input.

6. The differential pressure limiter of claim 1 wherein the first proportional valve includes a first proportional valve input and a first proportional valve output, the first proportional valve changing the fluid pressure of the cryogenic fluid at the first proportional valve output in a proportional manner to the fluid pressure of the cryogenic fluid at the first proportional valve input.

7. The differential pressure limiter of claim 1 wherein the backpressure regulator decreases the first differential fluid pressure of the cryogenic fluid across the first proportional valve to less than approximately 100 psi.

8. The differential pressure limiter of claim 1 wherein the backpressure regulator decreases the first differential fluid pressure of the cryogenic fluid across the first proportional valve to less than approximately 50 psi.

9. The differential pressure limiter of claim 1 wherein the backpressure regulator decreases the first differential fluid pressure of the cryogenic fluid across the first proportional valve to less than approximately 30 psi.

10. The differential pressure limiter of claim 1 wherein the backpressure regulator decreases the first differential fluid pressure of the cryogenic fluid across the first proportional valve to less than approximately 10 psi.

11. The differential pressure limiter of claim 1 wherein the backpressure regulator maintains the first differential fluid pressure of the cryogenic fluid across the first proportional valve within a predetermined range.

12. The differential pressure limiter of claim 1 further comprising a second proportional valve that is positioned between the pressure regulator and the backpressure regulator.

13. The differential pressure limiter of claim 1 wherein the backpressure regulator is manually adjustable to change the first differential fluid pressure of the cryogenic fluid across the first proportional valve.

14. The differential pressure limiter of claim 1 wherein the backpressure regulator is automatically adjustable to change the first differential fluid pressure of the cryogenic fluid across the first proportional valve.

15. A differential pressure limiter for limiting a differential fluid pressure of a cryogenic fluid that is delivered to a catheter assembly from a fluid source during a cryoablation procedure, the differential pressure limiter comprising:
a pressure regulator that receives the cryogenic fluid from the fluid source, the pressure regulator having a regulator output, the pressure regulator regulating a fluid pressure of the cryogenic fluid at the regulator output;
a first proportional valve that receives the cryogenic fluid from the regulator output of the pressure regulator, the cryogenic fluid having a first differential fluid pressure across the first proportional valve; and
a second proportional valve that at least partially controls a flow rate of the cryogenic fluid to the catheter assembly during the cryoablation procedure, the second proportional valve receiving the cryogenic fluid from the first proportional valve and changing the differential fluid pressure of the cryogenic fluid from the first differential fluid pressure to a second differential fluid pressure that is less than the first differential fluid pressure.

16. The differential pressure limiter of claim 15 wherein the second proportional valve decreases the second differential fluid pressure of the cryogenic fluid across the second proportional valve based on the first differential fluid pressure across the first proportional valve.

17. The differential pressure limiter of claim 15 further comprising a third proportional valve that at least partially controls the flow rate of the cryogenic fluid to the catheter assembly during the cryoablation procedure, the third proportional valve receiving the cryogenic fluid from the second proportional valve and changing the differential fluid pressure of the cryogenic fluid from the second differential fluid pressure to a third differential fluid pressure that is less than the second differential fluid pressure.

18. The differential pressure limiter of claim 17 wherein the second proportional valve is positioned between the first proportional valve and the third proportional valve.

19. The differential pressure limiter of claim 17 wherein the third proportional valve decreases the third differential fluid pressure of the cryogenic fluid across the third proportional valve based on the second differential fluid pressure across the second proportional valve.

20. A method, comprising the step of:
delivering a cryogenic fluid to a catheter assembly via a differential pressure limiter that is configured so that a proportional valve is positioned between a pressure regulator and a backpressure regulator to limit a differential fluid pressure of the cryogenic fluid across the proportional valve during a cryoablation procedure.
